(19) United States
(12) Patent Application Publication
(10) Pub. No.: US 2020/0238099 A1
(43) Pub. Date: Jul. 30, 2020

(54) METHOD OF USING A LIGHT-BASED DRUG DISPENSING SYSTEM TO SIMULATE THE OPERATION OF A HUMAN PANCREAS

(71) Applicants: Rama Madugula, Royal Oak, MI (US); Anup Sabharwal, Miami, FL (US); Sankar Majeti, Rochester Hills, MI (US); Murty Madugula, Rochester Hills, MI (US)

(72) Inventors: Rama Madugula, Royal Oak, MI (US); Anup Sabharwal, Miami, FL (US); Sankar Majeti, Rochester Hills, MI (US); Murty Madugula, Rochester Hills, MI (US)

(73) Assignee: Midwest Business Associates, Inc., Rochester Hills, MI (US)

(21) Appl. No.: 16/846,336

(22) Filed: Apr. 12, 2020

Related U.S. Application Data
(63) Continuation-in-part of application No. 15/975,973, filed on May 10, 2018.
Application No. 15/975,973 is a division of application No. 15/247,796, filed on Aug. 25, 2016, now abandoned.
Application No. 15/247,796 is a continuation of application No. 14/724,854, filed on May 29, 2015, now abandoned.

(51) Int. Cl. A61N 5/06; G16H 20/10; A61K 45/06; A61K 31/64; A61M 5/14; G08B 21/04

(52) U.S. Cl. CPC A61N 5/062; G16H 20/10; A61K 45/06; A61K 31/64; A61M 5/14; G08B 21/0453; A61M 2205/18; A61N 2005/0652; A61N 2005/0626; A61M 2205/583; A61M 2205/52; A61M 2205/3584; A61N 2005/0663

(57) ABSTRACT

Disclosed is a pump-less light-based biomedical apparatus and method for dispensing drug formulations and compounds to simulate the operation of a human pancreas without the drawbacks of an electromechanical pump-based design. Drug formulations dispensable by the method contain at least one photoswitchable molecular switch compound (such as azobenzene) whose structure and amounts of dosage released are controllable by light wavelength and intensity. Drug formulations are dispensed based on light generated by a set of LED arrays that includes blue LED's as directed by a closed-loop feedback control system within a control computer. The control system monitors multiple sensors, including a continuous blood glucose monitoring sensor, and performs control-system calculations based on sensor inputs. When necessary, the control system activates the LED arrays to cause the photoswitchable molecular switch compounds to be individually and selectively released in controlled amounts without the LED wavelengths interfering with each other.

### CROSS REFERENCE TO RELATED APPLICATIONS

The present invention is a continuation-in-part (CIP) of the U.S. patent application Ser. No. 15/975,973 (abandoned), filed 10 May 2018, entitled “Method of Using a Light-Based Drug Dispensing System to Simulate the Operation of a Human Pancreas” by Rama Madugula, et al., which is in turn a divisional of U.S. patent application Ser. No. 15/247,796 (abandoned), filed 25 Aug. 2016, entitled “Light-Based Drug Dispensing System to Simulate the Operation of a Human Pancreas”, by Rama Madugula, et al., which is, in turn, a continuation-in-part (CIP) of U.S. patent application Ser. No. 14/724,854 (abandoned), filed 29 May 2015, entitled “System for Medical Device Software Alert Hierarchy Management”, by Rama Madugula, et al.

### BACKGROUND OF THE INVENTION

### a. Field of the Invention

The present invention generally pertains to biomedical devices for diabetic patients, and more particularly to a solid-state wearable insulin medication delivery device with closed-loop feedback control that can function as an artificial pancreas.

### b. Description of the Background

Patients with diabetes mellitus suffer from a partial or total inability to produce insulin, resulting in poor control of their glucose variability. Type I diabetics do not produce any endogenous insulin and may require a wearable insulin pump from a young age. Type II diabetics have their ability to produce sufficient endogenous insulin degraded over time, may require oral or injectable medications for glucose control, and may eventually also need a wearable insulin pump.

Because the human pancreas releases many other compounds besides insulin, an artificial pancreas must be able to selectively release many other compounds besides insulin to approximate the function of a human pancreas. With Type II diabetes, it is desirable for patients, as much as possible, to use non-insulin medication to control glucose variability. This avoids long-term systemic effects of exogenous insulin on the patient while still keeping blood glucose within a target range.

Currently available wearable insulin pumps generally consist of a small wearable computer, a small electromechanical pump, a medication reservoir that holds only insulin, and an infusion set to connect the dispensed insulin to the patient's body. A control computer with the pump calculates the number of steps needed by a stepper motor to provide the calculated amount of insulin. The stepper motor converts these step commands into motion that moves insulin from the reservoir through the infusion set into the patient. Thus, the primary result of the control computer is the number of steps required by the stepper motor to maintain glucose control. Such electromechanical pumps have drawbacks: the pump must be calibrated, the minimum insulin dose is one turn of the stepper motor (even if less insulin is actually needed), the pump or infusion set can become clogged, and the moving parts can break down.

In recent years, a class of azobenzenes has been developed that change state, or are “photoswitchable”, when exposed to a specific wavelength of light. These azobenzenes may be combined with or located within other chemical compounds (including insulin, sulfonylureas, glucagon, dextrose, hormones, anti-inflammatories, peptides and other diabetic pharmaceutical compounds) that cause those compounds to be released and also to become photoswitchable.

Light-emitting diodes (LED's) are capable of delivering light at a specific wavelength and intensity. Thus, the intensity, wavelength, duration and duty-cycle of an LED light array can be controlled by a computer program to selectively release these photoswitchable drug formulations in precisely controlled dosages into the human body. This would allow diabetic medical compounds to be dispensed to a patient without using an electromechanical pump prone to failure.

It would be advantageous to have a solid-state device with no moving parts, such as an LED array, to release compounds, rather than to use an electromechanical pump that is prone to failure. It would also be advantageous if the solid-state device could selectively release any of several drug formulations, as an electromechanical pump can only release insulin. It would further be advantageous if the solid-state device could provide bidirectional blood glucose control, releasing compounds that lower blood glucose in periods of hyperglycemia or compounds that raise blood glucose in periods of hypoglycemia. Note that the insulin in an electromechanical pump only lowers glucose levels; if a patient's glucose level must be raised, the pump is of little use.

The present invention is a solid-state device that releases photoswitchable drug formulations in controlled doses into the body but has no moving parts. Sensor inputs, including a continuous blood glucose monitoring sensor (CGMS), allow calculation of light-control parameters to selectively dispense calculated dosages of drug compounds to a patient to control glucose variability. The device controls a reservoir of drug formulations placed inside the patient and avoids the clogging associated with connecting an external electromechanical pump to a patient via an infusion set, as can occur with available insulin pumps.

The present invention acts as a solid-state artificial pancreas, using a wearable computer, a control program using standard closed-loop feedback control algorithms (such as proportional-integral-derivative [PID], fuzzy logic control or model predictive control [MPC]), a CGMS, an LED light array, and photoswitchable drug formulations to keep a patient's blood glucose level within a range specified by a physician. Alerts are created when the blood glucose level is outside of the specified range, or when a system diagnostic requires user attention. The present invention includes a closed-loop feedback control algorithm in a control program within the wearable computer that requires a CGMS to measure blood glucose levels and send data back to the control program. The control program then uses the closed-loop feedback control algorithm to calculate the required amount of diabetic medical formulations to bring the patient's blood glucose back to the target range. The control program then converts that information into wavelength, intensity, duration and duty cycle parameters required to activate the LED array. The LED array then selectively releases one or more photoswitchable compounds, as needed, to bring the blood glucose level back to the target range.

After a period of time, the control program again takes data from the CGMS to close the loop and determine the blood glucose change, based on the dispensed medication amount. The control program then calculates any additional medication that needs to be dispensed and uses the LED array to dispense that additional medication. Using CGMS feedback, the control program does the calculation as many times as necessary to bring the patient's blood glucose levels back to the target range. The control program can dispense medical compounds in cases of hyperglycemia, as well as hypoglycemia, to keep blood glucose level within the specified range.

The present invention includes a closed-loop feedback control algorithm that requires a continuous glucose monitor to measure blood glucose levels and sends monitor data to a computer program that uses the closed-loop feedback control algorithm to govern an LED light array whose intensity, wavelength, duration and duty cycle is used to maintain glucose control by selectively releasing the photoswitchable compounds as needed to keep blood glucose levels within the specified range.

The present invention is an improvement on currently available computer-controlled pump-based insulin delivery devices because it eliminates the electromechanical pump with its attendant problems and limitations of moving parts. It can also deliver medical compounds other than insulin, thus allowing more pharmaceutical options to control blood glucose levels. It can deliver much more precise doses of medical formulations than a stepper motor, via precise pulse width modulation control of the LED array. Further, the present invention can provide glucose variability control in both hyperglycemic and hypoglycemic situations.

### SUMMARY OF THE INVENTION

The present invention overcomes the disadvantages and limitations of the prior art by creating a light-based solid-state apparatus with no moving parts that acts as an artificial pancreas to keep a diabetic patient's blood glucose level within a range specified by a physician. A wearable computer system that includes a closed-loop feedback control program is adjusted by a physician or technician and, using the control program to monitor the blood glucose level of the patient with the aid of a continuous blood glucose sensor, administers a medical compound to the patient, thus simulating a human pancreas, whenever said blood glucose level is outside of the range.

The apparatus includes: a set of sensors attached to the patient that, besides blood glucose level, monitors other health parameters; a drug reservoir that is supplied through a drug port with at least one drug formulation, designed to be given to the patient in controlled doses whenever his blood glucose level is outside of the range as registered by the control program, whose chemical structure and dosage amounts are controllable by light energy of a specified wavelength, intensity and duration; at least one set of LED arrays, each containing at least one LED that, when said LED is activated by the control program, generates at least one light beam of a wavelength, intensity and duration specified by the control program that controls the dosage of the light-controlled drug formulation being administered to the patient; a photoreceiver that alerts the wearable computer system whenever the set of LED arrays is activated but is not providing light; a display device that provides a visual alert, as directed by the control program, whenever the wearable computer system or said sensors is malfunctioning or whenever the blood glucose level is outside of the range; an auditory alarm that makes a sound whenever the wearable computer system or sensors is malfunctioning, or whenever the blood glucose level is outside of the range; and, a wireless message module that contacts the physician and other people as needed whenever the wearable computer system or sensors is malfunctioning, or whenever the blood glucose level is outside of the range.

The apparatus may have a set of sensors that includes a heart monitor, a sensor to monitor the body temperature of the patient, or a blood pressure sensor. The drug formulation may treat diabetes or be a sulfonylurea compound, insulin, glucagon, dextrose, a peptide, a hormone, an anti-inflammatory compound, or a diabetic pharmaceutical compound. The sulfonylurea compound may be JB253. The drug reservoir may be subcutaneous or intramuscular within the body of the patient, or within the visceral cavity of the patient. The computer program may control the set of LED arrays by setting the pulse width modulation duty cycle, or by setting the maximum amount of LED activation within a given time period. The set of LED arrays may be either located inside or outside the body of the patient. The set of LED arrays may contain LED's that are blue in color. Multiple drug formulations within the drug reservoir may be each controlled by a separate LED array operating at separate wavelengths, such that only one drug formulation can be released at a time to the patient, and such that the drug formulations, LED arrays, and wavelengths do not interfere with each other. The alerts generated for problems detected with the patient, wearable computer system or sensors may be sent to visual displays, auditory alarms, a message module, or wireless communications methods.

The present invention also discusses a method of keeping a diabetic patient's blood glucose level within a range specified by a physician, using a light-based solid-state apparatus with no moving parts as an artificial pancreas, comprising: monitoring the blood glucose level of a patient with a continuous blood glucose sensor; monitoring other health parameters of the patient, besides blood glucose level, with a set of sensors attached to the patient; using a wearable computer system, including a closed-loop feedback control program, to administer a light-controlled drug formulation to the patient whenever the blood glucose level is outside of the range; administering the light-controlled drug formulation to the patient, whenever the blood glucose level is outside of the range, from a drug reservoir that is supplied through a drug port and whose chemical structure and dosage amounts are controllable by light energy of a specified wavelength, intensity and duration; controlling the dosage of the light-controlled drug formulation by using the control program to activate at least one set of LED arrays, each containing at least one LED that, when activated, generates at least one light beam of a wavelength, intensity and duration as specified by the control program; using a photoreceiver to alert the wearable computer system whenever the set of LED arrays is activated but is not providing light; providing a visual alert, as directed by the control program, whenever the wearable computer system or sensors are malfunctioning, or whenever the blood glucose level is outside of the range; providing an auditory alert, as directed by the control program, that makes a sound whenever the wearable computer system or sensors are malfunctioning, or whenever the blood glucose level is outside of the range; and, using a wireless message module to contact the physician and other people as needed whenever the wearable computer system or sensors are malfunctioning, or whenever the blood glucose level is outside of said range.

This method may use a set of sensors that includes a heart monitor, a sensor to monitor the body temperature of the patient, or a blood pressure sensor. The drug formulation may treat diabetes or be a sulfonylurea compound, insulin, glucagon, dextrose, a peptide, a hormone, an anti-inflammatory compound, or a diabetic pharmaceutical compound. The sulfonylurea compound may be JB253. The drug reservoir may be subcutaneous or intramuscular within the body of the patient, or within the visceral cavity of the patient. The computer program may control the set of LED arrays by setting the pulse width modulation duty cycle, or by setting the maximum amount of LED activation within a given time period. The set of LED arrays may be either located inside or outside the body of the patient. The set of LED arrays may contain LED's that are blue in color. Multiple drug formulations within the drug reservoir may be each controlled by a separate LED array operating at separate wavelengths, such that only one drug formulation can be released at a time to the patient, and such that the drug formulations, LED arrays, and wavelengths do not interfere with each other. The alerts generated for problems detected with the patient, wearable computer system or sensors may be sent to visual displays, auditory alarms, a message module, or wireless communications methods.

### BRIEF DESCRIPTION OF THE DRAWINGS

In the drawings,

FIG. 1 is a drawing of a pump-based insulin system in prior art that uses an insulin pump and a stepper motor to deliver insulin to a diabetes patient.

FIG. 2 is a drawing of the light-based system of the present invention, which replaces the insulin pump and stepper motor of FIG. 1 with a light-based drug dispensing system for delivering multiple drug formulations, including insulin, to a diabetes patient.

FIG. 3 is an operational flowchart of the present invention.

### DETAILED DESCRIPTION OF THE INVENTION

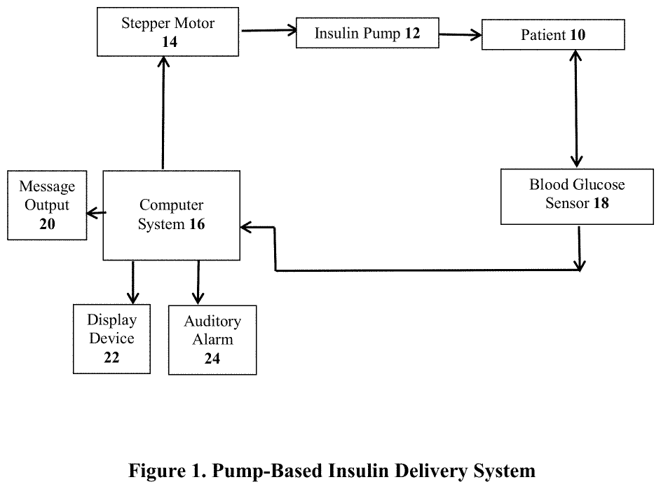

FIG. 1 is a drawing of a pump-based insulin system in prior art. A diabetic patient 10 receives insulin from an insulin pump 12 as driven by a stepper motor 14. The settings of the stepper motor 14 are controlled by a closed-loop feedback control program within a computer system 16 that adjusts the stepper motor 14 based on calculations by, and settings in, the closed-loop feedback control program to deliver insulin as needed from the insulin pump 12. The closed-loop feedback control program directs the computer system 16 to adjust the stepper motor 14 based on input from a variety of sensors, primarily a blood glucose sensor 18 that monitors the glucose levels of the patient 10 and provides that information to the computer system 16. The computer system 16 provides status updates to doctors and other people involved with the patient through a message output 20 and a display device 22 and sounds an auditory alarm 24 if the patient needs assistance.

Though the system in FIG. 1 is well-known, its imperfections are also well-known. The insulin pump 12 may clog, the stepper motor 14 may fail, and the stepper motor 14 must make at least one turn, creating a minimum insulin dose even if less insulin is actually needed. A light-based system with no moving parts that does not require an insulin pump or stepper motor would thus be an improvement over prior art.

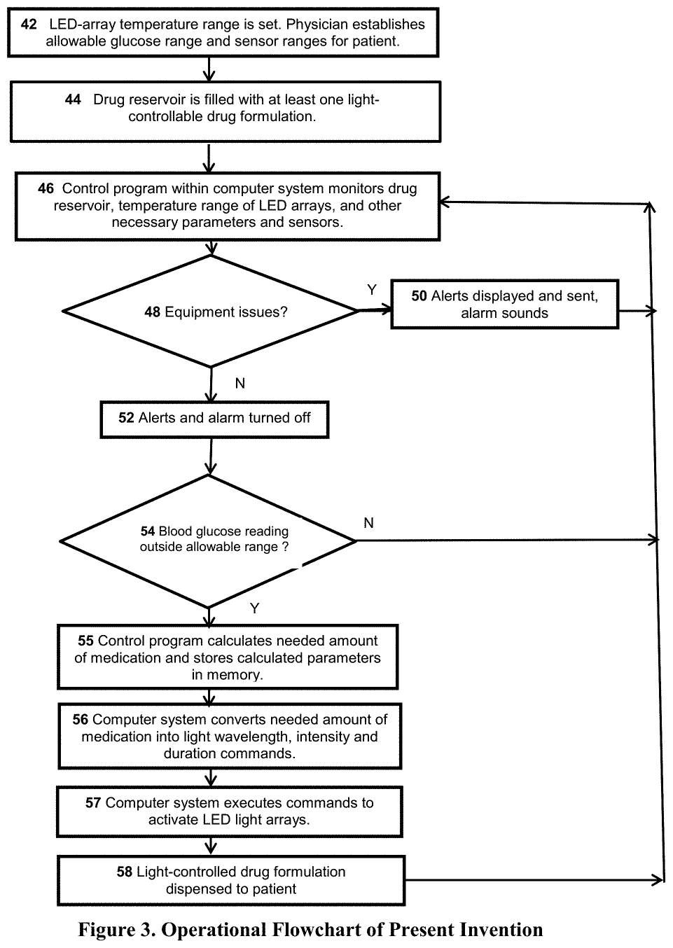

FIG. 2 is a drawing of the light-based system of the present invention, which replaces the insulin pump and stepper motor of FIG. 1 with a light-based drug delivery system for delivering multiple drug formulations, including insulin, to a diabetes patient.

A drug reservoir 26 is supplied with at least one light-controlled drug formulation 28 through a drug port 30. Each light-controlled drug formulation 28 in the drug reservoir 26 contains a photoswitchable compound whose chemical structure can be controlled with light by the computer system 16, using a closed-loop feedback control program within the computer system 16. That closed-loop feedback control program can activate a light-emitting diode (LED) within at least one set of LED arrays 32 to emit light of a specified wavelength. That wavelength, unique to each drug formulation 28, allows any desired drug formulation 28 in the drug reservoir 26 to be released into the body of the patient 10 in controlled doses via the light energy generated when the LED corresponding to that drug's light wavelength is activated by the closed-loop feedback control program.

If multiple drugs are present within the drug reservoir 26, there may exist as many unique LED light wavelengths as necessary so that only one drug formulation is released at a time to the patient 10. The drug reservoir 26 may be located either subcutaneously, intramuscularly, or within the visceral cavity of the patient 10.

A physician or technician may set the closed-loop feedback control program within the computer system 16 to provide feedback and control the sets of LED arrays 32 to generate light at specific wavelengths 34 that may be received by drugs within the drug reservoir 26. Sensors, including a temperature sensor 36, blood glucose sensor 18 as in FIG. 1, and other sensors 38 may be connected to the patient 10 either inside or outside the patient's body. All sensor outputs may be provided to the computer system 16, along with the outputs of the drugs within the drug reservoir 26. Both the drug reservoir 26 and sets of LED arrays 32 may be located either inside or outside the patient's body.

A physician or technician may also set the closed-loop feedback control program within the computer system 16 to control the sets of LED arrays 32 by setting the pulse width modulation duty cycle and the maximum amount of LED activation within a given time period. A physician or technician may also set the closed-loop feedback control program within the computer system 16 to monitor related parameters, including the temperature range of the LED arrays 32 as registered by the temperature sensor 36 and the range of glucose levels registered by the blood glucose sensor 18 at which all LED's in the LED arrays 32 should be deactivated. As a safety mechanism, a physician or technician may also set the closed-loop feedback control program within the computer system 16 to limit the total amount of time the LED arrays 32 may be activated within a defined period to protect a patient from an overdose of a light-controlled drug formulation 28.

Information from the computer system 16 may be viewed on a display device 22 and output to a wireless message module 40 to allow notification of the patient, family members and caregivers of any parameter outside established ranges. Any parameter outside established ranges may cause the computer system 16, as directed by the closed-loop feedback control program, to generate a visual alert to a display device 22 and an auditory alarm 24 that the system of FIG. 2 may need attention and possible intervention.

A photoreceptor 33 placed next to the LED array 32 lets the closed-loop feedback control program within the computer system 16 know that the LED array 32 is operating properly. If the LED array 32 is turned on and the photoreceiver 33 is not detecting light, then the closed-loop feedback control generates a visual alert to a display device 22 and an auditory alarm 24 that the LED array 32 may need attention and possible intervention.

In optimum embodiments of the present invention, the LED's in the LED arrays 32 may be blue in color, and each light-controlled drug formulation 28 may be controlled by a separate LED array 32 and different light wavelengths 34 such that the light-controlled drug formulations 28, LED arrays 32, and wavelengths 34 do not interfere with each other. The light-controlled drug formulations 28 may include (but are not limited to) a drug formulation to treat diabetes, a sulfonylurea compound (such as JB253), insulin, glucagon, dextrose, a peptide, a hormone, an anti-inflammatory compound, or a diabetic pharmaceutical compound. Dispensing of the light-controlled drug formulations 28 thus simulates the operation of a human pancreas.

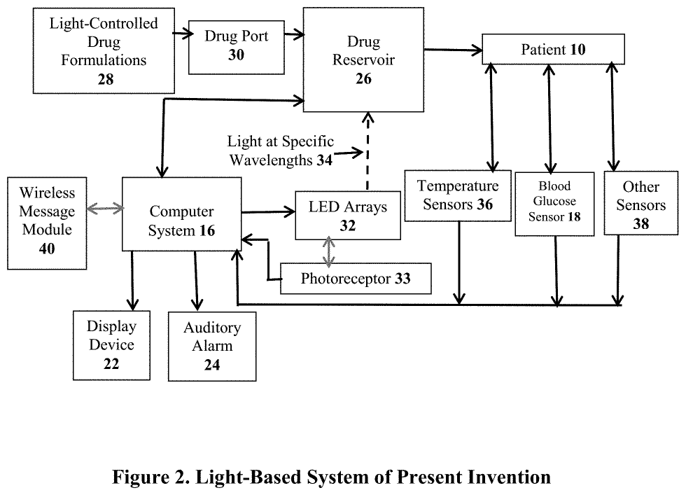

FIG. 3 illustrates an operational flowchart for the present invention. A range of allowable temperatures for the LED arrays 32 is established 42 such that the LED arrays 32 do not thermally injure the patient 10. A physician establishes an allowable range of high and low blood glucose levels 42 individualized for each patient, along with a range for any other sensors that may be used in the system (such as, but not limited to, a heart monitor, patient's body temperature sensor, or blood pressure sensor). All necessary light-controlled drug formulations 28 (referring to FIG. 2) are made available 44 to the patient 10 by placing the light-controlled drug formulations 28 in the drug reservoir 26. If all components shown in FIG. 2 are functioning properly, the LED arrays 32 remain within their given temperature range as monitored by the temperature sensor 36, the drug reservoir 26 contains sufficient quantities of the light-controlled drug formulations 28, and the patient's body temperature range, glucose level and other parameters are within normal ranges as detected by the blood glucose sensor 18 and other sensors 38 in the system, then the present invention remains in a loop 46, where the closed-loop feedback control program within the computer system 16 is continuously running to gather sensor data, perform control calculations, and run diagnostic tests.

Whenever any equipment in FIG. 2 fails to function properly 48, the closed-loop feedback control program within the computer system 16 detects the situation and, as necessary, sends an alert 50 to the display device 22 and the wireless message module 40 and an alert to the auditory alarm 24 to create a sound. These alerts continue until the equipment issue has been addressed, after which the alerts and alarm are turned off 52 by the closed-loop feedback control program within the computer system 16. Such equipment failures may include a lack of light to the light-controlled drug formulations 28 as noted by the photoreceiver 33, the drug reservoir 26 needing to be refilled with light-controlled drug formulations 28, or the LED array 32 being hot enough to thermally injure the patient as measured by the temperature sensor 36.

If all equipment in FIG. 2 is working properly and the blood glucose reading monitored by the closed-loop feedback control program within the computer system 16 detects a patient's glucose level 54 to be outside the allowable range 42 set by the physician, the closed-loop feedback control program within the computer system 16 calculates the amount of medication necessary to give the patient 10 and stores the related parameters in memory 55. The computer system 16 then converts the amount of medication needed into commands that direct one or more specific LED arrays 32 to generate light at a specified wavelength 34, as well as a specified intensity and duration 56, that corresponds to a single light-controlled drug formulation 28 within the drug reservoir 26.

The closed-loop feedback control program within the computer system 16 may use an established control algorithm (such as proportional-integral-derivative [PID], fuzzy logic control or model predictive control [MPC]) to execute commands 57 to activate the LED arrays 32, either duty-cycling the LED's with pulse width modulation or simply turning on the array for the calculated duration of time to generate the specified intensity of the light 34.

The specific wavelength of the light 34, as well as the light's intensity, activates a single light-controlled drug formulation 28 within the drug reservoir 26, which is then delivered 58 to the patient 10 in an appropriate dosage previously calculated by the physician and closed-loop feedback control program within the computer system 16, based on data provided by the blood glucose sensor 18 and other sensors 38.

The light-controlled drug formulation 28 may be administered 58 until the blood glucose sensor and other sensors indicate that the patient's blood glucose level and other levels are again within the preset “normal” range 42, at which point the closed-loop feedback control program within the computer system 16 sends a message to the activated LED arrays 32 that turns off the LED's and ends the dosage of the light-controlled drug formulation 28. The present invention may then return to the loop 46 where the closed-loop feedback control program continuously runs to monitor all sensors and perform control calculations, and the drug reservoir 26 may be replenished as needed.

In all embodiments of the present invention, alerts may be sent by common electronic communications methods, including wireless methods, that may include e-mail, texting, cell phone and Bluetooth. Such alerts may be seen on a remote device such as a smartphone, wrist watch or other display in a remote location and may notify all interested parties, such as a patient, caregiver, physician and hospital, of a patient's health issues.

The present invention reduces the cost of a patient's treatment regimen and improves a patient's long-term health while avoiding the insulin pump clogging, stepper motor failure and issues of imprecise control that exist with electromechanical systems similar to FIG. 1.

The foregoing description of the invention has been presented for purposes of illustration and description. It is not intended to be exhaustive or to limit the invention to the precise form disclosed, and other modifications and variations may be possible in light of the above teachings. The embodiments were chosen and described in order to best explain the principles of the invention and its practical application to thereby enable others skilled in the art to best utilize the invention in various embodiments and various modifications as are suited to the particular use contemplated. It is intended that the appended claims be construed to include other alternative embodiments of the invention except insofar as limited by the prior art.

What is claimed is:

1. A light-based solid-state apparatus with no moving parts that acts as an artificial pancreas to keep a diabetic patient's blood glucose level within a range specified by a physician, comprising:
a wearable computer system that includes a closed-loop feedback control program adjusted by said physician or by a technician and, using said control program to monitor the blood glucose level of said patient with the aid of a continuous blood glucose sensor, administers a medical compound to said patient, thus simulating a human pancreas, whenever said blood glucose level is outside of said range;
a set of sensors attached to said patient that, besides blood glucose level, monitors other health parameters of said patient;
a drug reservoir that is supplied through a drug port with at least one drug formulation, designed to be given to said patient in controlled doses whenever said blood glucose level is outside of said range as registered by said control program, whose chemical structure and dosage amounts are controllable by light energy of a specified wavelength, intensity and duration;
at least one set of LED arrays, each containing at least one LED that, when said LED is activated by said control program, generates at least one light beam of a wavelength, intensity and duration specified by said control program that controls the dosage of said light-controlled drug formulation being administered to said patient;
a photoreceiver that alerts said wearable computer system whenever said set of LED arrays is activated but is not providing light;
a display device that provides a visual alert, as directed by said control program, whenever said wearable computer system or said sensors is malfunctioning or whenever said blood glucose level is outside of said range;
an auditory alarm that makes a sound whenever said wearable computer system or said sensors is malfunctioning or whenever said blood glucose level is outside of said range; and,
a wireless message module that contacts said physician and other people as needed whenever said wearable computer system or said sensors is malfunctioning or whenever said blood glucose level is outside of said range.

2. The apparatus of claim 1, wherein said set of sensors includes a heart monitor, a sensor to monitor the body temperature of said patient, or a blood pressure sensor.

3. The apparatus of claim 1, wherein said drug formulation includes a drug formulation to treat diabetes, a sulfonylurea compound, insulin, glucagon, dextrose, a peptide, a hormone, an anti-inflammatory compound, or a diabetic pharmaceutical compound.

4. The apparatus of claim 3, wherein said sulfonylurea compound is JB253.

5. The apparatus of claim 1, wherein said drug reservoir is subcutaneous or intramuscular within the body of said patient, or within the visceral cavity of said patient.

6. The apparatus of claim 1, wherein said computer program may control said set of LED arrays by setting the pulse width modulation duty cycle or by setting the maximum amount of LED activation within a given time period.

7. The apparatus of claim 1, wherein said set of LED arrays is located inside the body of said patient or outside the body of said patient.

8. The apparatus system of claim 1, wherein said set of LED arrays contain LED's that are blue in color.

9. The apparatus of claim 1, wherein multiple drug formulations within said drug reservoir are each controlled by a separate LED array operating at separate wavelengths such that only one drug formulation can be released at a time to said patient, and such that said drug formulations, said LED arrays, and said wavelengths do not interfere with each other.

10. The apparatus of claim 1, wherein the alerts generated for problems detected with said patient, said wearable computer system or said sensors are sent to visual displays, auditory alarms, a message module, or wireless communications methods.

11. A method of keeping a diabetic patient's blood glucose level within a range specified by a physician, using a light-based solid-state apparatus with no moving parts as an artificial pancreas, comprising:
monitoring the blood glucose level of said patient with a continuous blood glucose sensor;
monitoring other health parameters of said patient, besides blood glucose level, with a set of sensors attached to said patient;
using a wearable computer system, including a closed-loop feedback control program, to administer a light-controlled drug formulation to said patient whenever said blood glucose level is outside of said range;
administering said light-controlled drug formulation to said patient, whenever said blood glucose level is outside of said range, from a drug reservoir that is supplied through a drug port and whose chemical structure and dosage amounts are controllable by light energy of a specified wavelength, intensity and duration;
controlling the dosage of said light-controlled drug formulation by using said control program to activate at least one set of LED arrays, each containing at least one LED that, when activated, generates at least one light beam of a wavelength, intensity and duration as specified by said control program;
using a photoreceiver to alert said wearable computer system whenever said set of LED arrays is activated but is not providing light;
providing a visual alert, as directed by said control program, whenever said wearable computer system or said sensors are malfunctioning or whenever said blood glucose level is outside of said range;
providing an auditory alert, as directed by said control program, that makes a sound whenever said wearable computer system or said sensors are malfunctioning or whenever said blood glucose level is outside of said range; and,
using a wireless message module to contact said physician and other people as needed whenever said wearable computer system or said sensors are malfunctioning or whenever said blood glucose level is outside of said range.

12. The method of claim 11, wherein said set of sensors attached to said patient includes a heart monitor, a sensor to monitor the body temperature of said patient, or a blood pressure sensor.

13. The method of claim 11, wherein said light-controlled drug formulation is a drug formulation to treat diabetes, a sulfonylurea compound, insulin, dextrose, glucagon, a hormone, a peptide, an anti-inflammatory compound, or a diabetic pharmaceutical compound.

14. The method of claim 13, wherein said sulfonylurea compound is JB253.

15. The method of claim 11, wherein said drug reservoir is subcutaneous or intramuscular within the body of said patient, or within the visceral cavity of said patient.

16. The method of claim 11, wherein said computer program may control said set of LED arrays by setting the pulse width modulation duty cycle or by setting the maximum amount of LED activation within a given time period.

17. The method of claim 11, wherein said set of LED arrays is located inside the body of said patient or outside the body of said patient.

18. The method of claim 11, wherein said set of LED arrays contain LED's that are blue in color.

19. The method of claim 11, wherein multiple drug formulations within said drug reservoir are each controlled by a separate LED array operating at separate wavelengths such that only one drug formulation can be released at a time to said patient, and such that said drug formulations, said LED arrays, and said wavelengths do not interfere with each other.

20. The method of claim 11, wherein the alerts generated for problems detected with said patient, said computer system or said sensors are sent to visual displays, auditory alarms, a message module, or wireless communications methods.
